(19) United States
(12) Patent Application Publication
(10) Pub. No.: US 2024/0358978 A1
(43) Pub. Date: Oct. 31, 2024

(54) CATHETER WITH INTEGRATED DISPENSER AND RELATED METHOD OF MANUFACTURE

(71) Applicant: B. BRAUN MELSUNGEN AG, Melsungen (DE)

(72) Inventor: Hui Kuun Teoh, Penang (MY)

(73) Assignee: B. BRAUN MELSUNGEN AG, Melsungen (DE)

(21) Appl. No.: 18/574,282

(22) PCT Filed: Jun. 28, 2022
(86) PCT No.: PCT/EP2022/067751

§ 371 Date: Dec. 26, 2023

Related U.S. Application Data
(60) Provisional application No. 63/216,265, filed on Jun. 29, 2021.

(51) Int. Cl. A61M 25/09; A61M 25/00; A61M 25/01; A61M 25/06

(52) U.S. Cl. CPC A61M 25/09041; A61M 25/0009; A61M 25/0097; A61M 25/0606; A61M 25/0612; A61M 2025/0177; A61M 2025/09116; A61M 2025/09125; A61M 2205/3331; A61M 2205/58; A61M 2205/75

(57) ABSTRACT

A catheter assembly having a catheter unit with a catheter hub having a hub body and a catheter tube and an integrated needle unit having a needle hub with a body and having a needle with a needle lumen extending out a distal end of the needle hub. The needle hub has aa distal path having a distal path opening and having the needle attached to an end of the distal path, a proximal path having a proximal path opening and a rear-most opening, and a working surface on a working platform located between the distal path opening and the proximal path opening. A guidewire can optionally pass between the distal path and the proximal path and cause to dispenser when pressed against the working surface.

### FIELD OF ART

The disclosed invention generally relates to needle devices and intravenous (IV) infusion devices, including IV catheters, peripheral catheters, and midline catheters. In particular, IV catheter assemblies having a one-handed operation for dispensing guidewires are disclosed.

### BACKGROUND

IV catheters are commonly used for a variety of infusion therapies, including infusing fluids into a patient, withdrawing blood from a patient, or monitoring various parameters of the patient's vascular system. Catheters are typically connected to a catheter adapter that accommodates the attachment of IV tubing to the catheter. Blood control catheters include an internal blood control valve that is opened by the insertion of a male Luer or another object into a proximal end of the catheter adapter. Non-limiting examples of blood control valves are disclosed in United States Patent Application Publication No. 2011/0046570, filed Aug. 20, 2009, titled “Systems and Methods for Providing a Flushable Catheter Assembly.” Following placement of the catheter into the vasculature of a patient, an IV fluid source can be connected to the catheter adapter or catheter hub, opening the blood control valve. Thus connected, fluid from the IV source can begin flow into a patient through the catheter.

As is well known in the art, typical blood pressure is 10 to 20 centimeters of water. Infusion bags are usually placed about 100 cm above the patient's heart to direct flow into the patient. At roughly that height, the pressure exerted by the fluid from the infusion bag is much greater than the blood pressure of the patient and therefore can flow into the patient.

For patients with difficult to access veins, extended dwell catheters can be used to aid in Difficult Intravenous Access (DIVA). An extended dwell catheter is a midline catheter that may be considered a peripherally inserted catheter. The recommended insertion site for the midline catheter is in the upper arm with the tip located just below the axilla. For DIVA patients, a physician can use visualization equipment to aid in identification of deep veins for catheter access. In that case, an extended dwell catheter will provide a longer length and a more flexible catheter for insertion into the patient. With the addition of a guidewire, this can help to reduce the chances of the catheter becoming kinked.

### SUMMARY

The various embodiments of catheter assemblies have several features, no single one of which is solely responsible for their desirable attributes. Embodiments of the present catheter assemblies include a catheter hub mounted to an integrated needle hub for carrying a hollow needle and a guidewire. Without limiting the scope of the present embodiments as expressed by the claims that follow, their more prominent features will now be discussed briefly. After considering this discussion, and particularly after reading the section entitled Detailed Description, one will understand how the features of the present embodiments provide advantages, which include an integrated design that allows for peripheral vascular access using traditional IVC procedures, among others.

Catheter assemblies are described. In an example, a catheter assembly is disclosed comprising a catheter unit comprising a catheter hub having a hub body with a proximal opening and having a catheter tube extending from a distal end of the catheter hub, the catheter tube having a catheter tube opening; an integrated needle unit comprising a needle hub with a body and having a needle with a needle lumen extending out a distal end of the needle hub, wherein the needle hub comprises: a distal path having a distal path opening and having the needle attached to an end of the distal path; a proximal path having a proximal path opening and a rear-most opening; a working surface on a working platform located between the distal path opening and the proximal path opening; and wherein the working surface is exposed from a side of the body.

The catheter assembly can further comprise a nose section on an end of the needle hub projecting into the proximal opening of the catheter hub in a ready to use position.

The catheter assembly may be used with or without a guidewire, like a standard peripheral intravenous catheter (PIVC).

A guidewire can extend through both the distal path, the proximal path, and at least part of the needle lumen in a ready to use position.

A tubing having a first end can couple directly to the needle hub.

The needle hub can have a socket and a second end of the tubing coupled to the socket.

The first end of the tubing can project into the rear-most opening of the needle hub.

The guidewire can locate partly inside the tubing. For example, the proximal end of the guidewire can extend into the hollow interior of the tubing.

The tubing can have a first end attached to the needle hub and a second end that is unsupported or is coupled to a socket located on the needle hub.

The guidewire can have a proximal end or second end and wherein the proximal end can be sized and shaped to abut the rear-most opening of the needle hub to stop distal movement of the guidewire. The proximal end of the guidewire can have an enlarged element, such a sleeve, a material buildup or a crimp, or can have a J-loop or a coiled loop.

The guidewire can have an enlarged section and wherein the enlarged section is sized and shaped to abut a crimp on the needle from inside the needle to stop distal movement of the guidewire.

The needle hub can have a first proximal path section and a guide stub extending from a proximal end surface of the needle hub and a second proximal path section aligned with the first proximal path section.

The needle hub can have a receiving cavity that bisects the distal path, or the distal path can pass through or originate at the receiving cavity.

A flow restrictor can be located in the receiving cavity.

The flow restrictor can be a septum, a valve, or a hydrophobic filter.

The needle hub can be sized and shaped to be gripped with one hand and the working surface of the working platform can be accessible by at least one of the fingers of the one hand.

The working surface can have an arc-shape between the proximal path and the distal path.

The needle hub can have a bottom surface and a thickness defined by the working surface and the bottom surface.

The needle hub can have a channel having the working platform and wherein sidewalls extend upwardly from the working surface of the working platform.

A needle guard can be located in an interior of the catheter hub, said needle guard can comprise two arms biased outwardly by the needle in the ready to use position.

A valve and a valve opener can be located in the interior of the catheter hub.

The valve can comprise a plurality of flaps and the needle guard is located between two plunger elements of the valve opener.

A tubing can attach to the needle hub and wherein a guidewire can be partly located inside the tubing and partly located inside the needle.

The guidewire can pass through both the distal path and the proximal path.

Aspects of the invention further includes a method of manufacturing a catheter assembly. The method can comprise the step of forming a catheter unit comprising a catheter hub having a hub body with a proximal opening and having a catheter tube extending from a distal end of the catheter hub, the catheter tube having a catheter tube opening; forming an integrated needle unit comprising a needle hub with a body and having a needle with a needle lumen extending out a distal end of the needle hub, wherein the needle hub comprises: a distal path having a distal path opening and having the needle attached to an end of the distal path; a proximal path having a proximal path opening and a rear-most opening; a working surface on a working platform located between the distal path opening and the proximal path opening; and wherein the working surface is exposed from a side of the body.

The method can further comprise projecting a nose section on an end of the catheter hub into the proximal opening of the catheter hub in a ready to use position.

The method can further comprise passing a guidewire through the proximal path, through the distal path, and at least partly into the needle lumen.

The method can further comprise exposing the guidewire at the working surface between the proximal path and through the distal path for pushing by a user.

The method can further comprise attaching a tubing to the needle hub before passing the guidewire through the proximal path.

The method can further comprise placing a flow restrictor into a receiving cavity formed at a distal end of the needle hub.

Aspects of the invention further includes a method of using a catheter assembly. The method of use can comprise gripping the catheter assembly with one hand to grip the catheter assembly with a holding hand, the catheter assembly comprising: a catheter unit comprising a catheter hub having a hub body with a proximal opening and having a catheter tube extending from a distal end of the catheter hub, the catheter tube having a catheter tube opening; an integrated needle unit comprising a needle hub with a body and having a needle with a needle lumen extending out a distal end of the needle hub, wherein the needle hub comprises: a nose section projecting into the proximal opening of the catheter hub in a ready to use position; a distal path having a distal path opening and having the needle attached to an end of the distal path; a proximal path having a proximal path opening and a rear-most opening; a working surface on a working platform located between the distal path opening and the proximal path opening; and wherein the working surface is exposed from a side of the body; orienting a finger of the holding hand over the working platform.

The method can further comprise pushing down on the guidewire with the finger to press the guidewire between the finger and the working surface.

Aspects of the invention include a catheter assembly comprising a catheter unit and a needle unit. The catheter unit can comprise a catheter hub and a catheter tube extending from the catheter hub.

The needle unit can comprise a needle hub having a needle attached thereto and the needle extending in a distal direction through the catheter hub and the lumen of the catheter tube with the needle tip projecting distally of the distal opening of the catheter tube in a ready to use position.

The catheter hub can have a hub body having a proximal opening with a female Luer receiving a distal end of the needle hub. The hub body can have a plurality of sides connected to one another along connecting corners or can have a traditional frusto-conical body, as further discussed below. A push tab can locate on the upper portion of the hub body for use by a practitioner to push on to separate the catheter hub from the needle hub following successful venipuncture.

A nose section can locate at the distal end of the hub body and can have a smaller cross-sectional dimension than the proximal portion of the hub body, such as by providing one or more tapered surfaces between the proximal end and the distal end of the hub body. Optionally, the nose section can be omitted. For example, a flange, tab, or arm can extend from the needle hub to secure against the catheter hub without using a nose section. The hub body may be made from traditional plastic materials used for catheter hubs with a translucent appearance.

The needle hub can comprise a body having a length and a width. The body can have a generally polygonal shaped cross-section with a plurality of sides that include a working platform, an irregular shaped cross-section with a working platform, or a generally circular shaped cross-section with a working platform.

The working platform can be defined by an upper surface or working surface that a user or practitioner of the catheter assembly can push at least one of his or her fingers, such as the index finger, against to dispense a guidewire, as further discussed below. The needle hub can have a nose section that projects into the interior of the catheter hub in the ready to use position. The catheter hub can have a female Luer at the proximal end for receiving the nose section of the needle hub. The catheter hub can also optionally incorporate a needle guard, a valve and/or a valve opener.

In an example, the body of the needle hub comprises a distal path and a proximal path. The two paths can be enclosed and each comprising a passage or bore or can comprise open channels. The two paths can be arranged or aligned to pass a guidewire therethrough or therebetween. From the working platform, the guidewire can have a length that extends in the distal direction, through the distal path, through the lumen of the needle, and having a guidewire distal end that terminates proximal of the needle tip opening.

From the working platform, the guidewire can have a length that extends in the proximal direction and out through the proximal path of the needle hub. The needle hub can incorporate a guide stub extending from the proximal end surface of the body to extend the length of the proximal path. The guide stub, when incorporated, has a bore aligned and in communication with the proximal path that originates at the working platform. The guide stub can be omitted from the body and an opening can be provided at the proximal end surface of the body for the guidewire to exit in the proximal direction.

A tubing having a length with a first end or distal end can engage the opening or bore of the guide stub located at the proximal end of the catheter hub, such as in an interference fit. Optionally, adhesive or glue may be used to more permanently secure the distal end of the tubing in the bore of the guide stub. The tubing can be flexible and be made from any number of prior art tubing materials. The tubing can be hollow or has a lumen and provided to store or accommodate the proximal part of the guidewire therein, the part that extends in the proximal direction out through the proximal path. The proximal end of the tubing can be supported. For example, the proximal end can connect to a socket on the needle hub body, which can be an open socket with a gap or a closed socket without a gap.

By engaging the socket, the tubing can coil into a loop or a partial loop to organize the tubing and the guidewire located therein, such as to keep the tubing and the guidewire from dangling out the proximal end of the needle hub. In other examples, the tubing can be unsupported at the proximal end of the tubing. For example, the tubing can extend from the needle hub without engaging the needle hub at the second end of the tubing.

As shown, the guidewire has a length and a proximal end that is located inside the lumen of the tubing. How far or long does the guidewire extend into the tubing can depend on how much of the guidewire is permitted to be dispensed out the needle tip, as further discussed below. In an example, the overall length of the guidewire is selected so that upon being dispensed by the user, the proximal end of the guidewire terminates within the working platform, which signifies the end of the guidewire length and therefore the guidewire cannot physically be dispensed any further.

In other examples, the guidewire can be provided with an enlarged section, such as an integrated notch, bump, build-up, a J-loop proximal end, or a sleeve, and upon dispensing the guidewire, the enlarged section is physical stopped by a relatively smaller opening formed with the needle hub, such as with the guide stub, the body, the distal path, or the proximal path. In still other examples, the needle can have a crimp and wherein distal advancement of the guidewire is stopped when the enlarged section of the guidewire contacts the crimp from inside the needle lumen.

The guidewire used herein is slidable or movable within the tubing when using a finger, such an index finger, to press the guidewire against the surface of the working platform and simultaneously sliding the guidewire against the surface in the distal direction to dispense the distal end of the guidewire out the needle tip.

As the needle unit carries both the needle and the guidewire without a separate guidewire dispenser having a guidewire dispenser housing, the needle unit can be referred to as an integrated needle and guidewire hub and the catheter assembly can be referred to as a catheter assembly with an integrated needle and guidewire hub.

In an example, a plurality of spaced apart surface features can be provided on one or more sides of the needle hub. The space apart surface features can embody bumps, recesses, or a combination of bumps and recesses of any number of shapes. As shown, the spaced apart surface features are bumps having an oblong shape or an oval shape with other shapes contemplated, such as a round shape, a square shape, a star shape, an irregular shape, etc. The surface features can improve gripping or can simply serve as aesthetic features.

The catheter assembly of the present invention, with a guidewire, can be operated using a one-handed technique. In an example, the hand grips the needle unit in a supinated position with the index finger located above or over the working platform to advance the guidewire following vascular access with the needle tip. The figure shows the hand being a right-hand, which is exemplary only as the user can optionally use the catheter assembly with a left-hand.

The tubing can be coiled below the hand with the first end or distal end coupled to the guide stub and the second end or proximal end of the tubing supported by a socket. A section of the guidewire, including the proximal end of the guidewire, can be located inside the lumen of the tubing and freely slidable inside the tubing when advanced by the user.

In an alternative embodiment, the first end of the tubing is attached to the needle unit but wherein the second end is spaced or not attached to the needle unit, such as being unsupported. The tubing may be generally straight as shown by incorporating a thicker, stiffer or a relatively higher durometer tubing material, or the tubing can bend or curved as it extends from the needle unit by incorporating a thinner, softer, or relatively lower durometer tubing material.

A guidewire can extend into the lumen of the tubing and the second end or proximal end of the guidewire can be located inside the tubing lumen, but extending short of the proximal opening at the second end of the tubing. The guidewire may be of a peripheral type, typically with a core diameter of about 0.014-in, 0.018-in, or 0.035-inch, to name a few non-limiting examples, and can be made from any number of prior art materials, such as stainless steel, nitinol, or a hybrid type.

A catheter unit provided in accordance with the invention can have a catheter hub and a catheter tube extending therefrom. The present catheter hub can embody a more traditional elongated generally round, generally frustoconical, hub body. The catheter hub can comprise a nose section having the catheter tube extending therefrom and a proximal opening receiving the nose section of the needle hub in the ready to use position. Internally of the catheter hub, a needle guard for covering the needle tip and/or a combination of a valve and valve opener can be positioned for controlling fluid flow through the catheter hub, as further discussed below.

The catheter unit can have a body with a proximal opening having a female Luer having the nose section of the needle hub located therein. An optional tab or flange can be provided adjacent the nose section to grip part of the catheter hub therebetween. The proximal opening of the catheter hub has exterior threads and may be referred to as a female threaded Luer.

The catheter hub has a body defining an interior cavity. The catheter tube extends distally of the nose section of the catheter hub and can be held to the catheter hub by a metal bushing or a ferrule. A valve or septum can be located in the interior cavity between a distal shoulder and a proximal shoulder formed on the interior surface of the catheter hub to retain the valve therebetween.

The valve can have a plurality of slits defining a plurality of flaps. The flaps can open to permit flow through the catheter hub or close to restrict or limit flow through the catheter hub. A valve opener or septum opener, also referred to as an actuator, having a nose section can be located proximally of the valve and inside a space defined by the skirt of the valve. The nose section of the valve opener can be generally cylindrical and has a bore having the needle passing therethrough, which has the guidewire located in the lumen thereof. One or more openings can be provided through the body of the nose section for fluid flushing.

The valve opener can have a plunger element extending proximally from the nose section. In an example, two plunger elements can extend from the nose section and having a gap therebetween for accommodating a needle guard, which can be located in the gap between the two plunger elements. A pair of bands or stabilizers can be provided with the valve opener. In an example, each band or stabilizer connects to both of the two plunger elements so that a continuous ring or loop is formed at the two bands and the two plunger elements. The ring or loop is spaced from the nose section. Through holes or through openings can be provided with the valve opener between the nose section and the ring. In an example, at least two through holes are present between the nose section and the ring.

A needle guard provided herein can include a proximal wall with a perimeter defining an opening for accommodating the needle passing through the proximal wall. A pair of arms can extend distally of the proximal wall with each arm having a distal wall and a curved lip at an end thereof. An elbow is defined between the arm and the distal wall of each arm and the two elbows define a maximum guard dimension when the two arms are biased outwardly by the needle in the ready to use position. The needle guard can be positioned at the gap between the two plunger elements with the proximal wall located proximally of the ring and the two elbows located distally of the ring, in the two through openings of the valve opener. In an example, the two elbows can contact the interior of the catheter hub, can be spaced from the interior of the catheter hub, can contact one or both of the two bands or stabilizers, can be spaced from the two bands, or combinations thereof in a ready to use position.

In use, when the needle retracts in the proximal direction following successful venipuncture and the needle tip moves proximally of the two distal walls of the needle guard and the two arms no longer biased outwardly by the needle, the two arms can recoil or move inwardly so that the profile at the two elbows is now smaller than the inside diameter of the continuous ring of the actuator, and a crimp on the needle can abut or contact the perimeter defining the opening on the proximal wall to then pull the needle guard out of the catheter hub along with the needle.

The valve and the valve opener can remain with the catheter hub to control fluid flow through the catheter hub. For example, a male Luer tip, such as a syringe tip, can be inserted into the female Luer to advance the valve opener distally to then open the valve with the nose section of the valve opener. The valve can have a plurality of slits defining a plurality of flaps that deflect by the nose section of the valve opener when the valve opener is advanced distally. In an example, the valve has three slits defining three flaps. The needle guard and the combination valve and valve opener, or both, can be incorporated with other catheter hubs described elsewhere herein.

Catheter assemblies described herein can be used with a guidewire using a one-handed technique to perform a puncture and the onto dispensing the guidewire, with the same gripping or operating hand. The process can start with gripping the catheter assembly with one hand in a supinated position. The hand used to grip and hold the catheter assembly may be referred to as the holding hand, which can be a left hand or a right hand. The user can then poke the patient to access the vein with the needle tip and catheter tube opening of the catheter tube. Proper needle puncture can be confirmed via primary blood flashback. Alternatively or additionally, an ultrasound probe or meter, such as a 2D or 3D ultrasound probe, may be used to verify proper needle puncture. If used, the ultrasound probe may be held with the free hand, the one without the catheter assembly. In some examples, the needle used can be selected to have the desired stiffness to minimize bending, particularly if the needle is longer than a standard peripheral intravenous catheter (PIVC). A sufficiently sized gauge needle, a ribbed needle, a dual diameter needle, or a multi-diameter needle may be used.

Next, the user can advance the guidewire while steadily holding the catheter assembly in the same holding hand roughly in the same position to limit or avoid moving the needle tip inside the vein from the targeted area. The user can advance the guidewire by using one of the fingers, such as the index finger, on the same hand that is holding the catheter assembly to press the guidewire against the surface of the working platform and then using friction between the fingertip and the surface incrementally advancing the wire by moving the finger between a proximal position on the working platform to a distal position, and then lifting the finger back to the proximal position on the working platform and repeat.

The guidewire can advance until a physical stop is activated, felt or triggered, such as when a crimp, a bump, or an enlarged section on the guidewire abuts the crimp on the needle from the inside of the needle, until the end of the guidewire is reached and no further guidewire is available for advancing, until the enlarged section on the guidewire abuts a narrow opening on the needle hub, in the tubing, or combinations thereof.

After the guidewire is advanced into the vein, the catheter tube can then slide or advance over the guidewire and the guidewire serves to support and guides the catheter tube into the vein. In an example, the catheter tube can advance over the guidewire by using one of the fingers of the holding hand to push the push tab on the catheter hub in the distal direction. This action can cause the catheter hub to separate from the needle hub. The separation can continue until the needle, the guidewire, and the needle hub are completely separated from the catheter hub and the tip of the catheter tube remaining inside the vein for vascular access. Thus, in using the catheter assembly of the present invention through the various steps, the user can use the same holding hand to manipulate the catheter assembly without having to change hand or to use both hands.

The catheter unit of the present invention can optionally have a needle guard for covering the needle tip in a protective position of the needle. In addition to or alternatively, the catheter unit can have a valve or septum and a valve opener or actuator. When incorporated, the distal walls of the needle guard can each have a vertical slit sized to accommodate the diameter of the guidewire but not the needle. The slits allow the distal walls to move distal of the needle tip in the protective position and accommodate the guidewire that extends out of the needle as the needle is withdrawn following successful venipuncture. When incorporated, the valve or septum and the valve or septum opener remain with the catheter hub for controlling flow through the catheter hub.

The catheter assembly of the present invention may be used in the same manner as a standard peripheral intravenous catheter (PIVC) assembly. That is, the initial puncturing of the patient and the separation of the needle and needle hub from the catheter hub following successful venipuncture involve essentially the same techniques, which can be performed using a one-handed technique. Additionally, the step of advancing the guidewire does not require switching the holding hand to the other hand, using both hands, or separating the holding hand from the needle hub. Minimizing hand changing or handling locations of the catheter assembly can reduce undesirable needle movement as the needle enters the vein and/or as the guidewire is advanced into the vein to guide the catheter tube. These benefits provide for easy adaptation and acceptance by practitioners of the disclosed catheter assemblies.

The catheter assemblies with integrated guidewire dispensing needle hub of the present invention can have at least one less attachment point normally found between the needle hub and a guidewire dispenser housing. Thus, as prior art needle hubs and guidewire dispenser housings can be provided in separate packages, the present catheter assembly can reduce at least one pre-procedure assembly step since there is no separate guidewire dispenser to attach to the needle hub. The present catheter assembly with integrated guidewire dispensing needle hub can also be practiced without an outer removable or separable housing used to guide the catheter hub during separation of the catheter hub from the needle hub typically found in midline catheters.

Still further, the present catheter assembly design, including the disclosed needle unit with an integrated guidewire and needle hub, can be used without a guidewire and used as a standard peripheral intravenous catheter (PIVC) assembly. For example, the catheter assembly as shown and described can be used without a guidewire.

The catheter assembly of the present invention can have a tubing length extending out from the needle hub and unsupported at the unattached end of the tubing. This adaptation can be used to show movement of the guidewire, particularly when the tubing is transparent or semi-opaque. The same concept applies when the tubing is supported at the proximal end of the tubing. The same concept applies equally when the catheter unit has a generally cylindrical, generally frusto-conical, shaped body.

The guidewire proximal end can be coiled to increase the overall size or profile of the guidewire at the proximal end to define an enlarged end. The enlarged end having an enlarged profile relative to the guidewire diameter at the proximal end can be used to engage or be trapped by the needle hub, such as by a small opening in the guide stub if incorporated or by a small opening in the main body of the needle hub or the tubing. In an example, the enlarged end can be a J-shaped tip or a coiled tip. The needle hub, such as the guide stub or the proximal path, can have a bore for accommodating the diameter of the guidewire but too small for the enlarged end of the guidewire to pass therethrough. Thus, when the guidewire is dispensed during use, engagement between the enlarged end and the structure defining the bore can stop further distal advancement of the guidewire, which correspondingly stops distal advancement of the guidewire tip further distally relative to the needle tip and therefore defines the dispensed length of the guidewire.

In alternative embodiments, the guidewire can have a bump or an enlarged section, such a material build-up, that is larger than the internal path through the lumen of the needle at the crimp section of the needle, near the needle tip. Upon being dispensed, the bump or build-up on the guidewire can engage the crimp on the needle to stop distal advancement of the guidewire. The crimp on the needle can thus be used to both engage and retract a needle guard and stop further distal advancement of the guidewire.

In use, a user can grip the catheter assembly with one hand in a supinated position. The user can hold the catheter assembly with the holding hand, which can be a left hand or a right hand, and an ultrasound probe in the other hand, referred to as the probe hand for holding an ultrasound probe. The user can then poke the patient using the holding hand to access the vein with the needle tip and the catheter tube opening. The user can use the ultrasound probe with the probe hand to monitor the puncture site for proper placement of the needle tip. Optionally, proper needle puncture can be confirmed via primary blood flashback in addition to verification with the ultrasound probe.

Next, the user can advance the guidewire out the needle while steadily holding the catheter assembly in the same holding hand and roughly in the same position to limit or avoid moving the needle tip inside the vein from the targeted area. The user can advance the guidewire by using one of the fingers, such as the index finger, on the same holding hand that is holding the catheter assembly to press the guidewire against the surface of the working platform and then using friction between the fingertip and the surface to incrementally advance the wire by moving the finger between a proximal position on the working platform to a distal position, and then lifting the finger back to the proximal position on the working platform and repeat. The guidewire can advance under review or monitor of the ultrasound probe held in the probe hand until physically stopped by an engagement between the guidewire and the needle hub or between the guidewire and the crimp on the needle, as previously discussed.

After the guidewire has been advanced into the vein, the catheter tube can then slide over the guidewire and the guidewire serves to support and guides the catheter tube into the vein. In an example, the catheter tube can advance over the guidewire by using one of the fingers of the holding hand to push the push tab on the catheter hub in the distal direction. This action causes the catheter hub to separate from the needle hub. Further catheter tube advancement can be monitored by the ultrasound probe, such as a 2D or a 3D ultrasound, held in the probe hand or non-holding hand. Thus, in moving through the different stages of gaining vascular access, the user can use the same holding hand to manipulate the catheter assembly without having to change hand or to use both hands.

The user can continue separating the needle, the guidewire, and the needle hub by moving the needle hub and the catheter hub away from one another until the needle hub, needle, and guidewire are completely separated from the catheter hub. After complete separation, the tip of the catheter tube remains inside the patient's vein for vascular access. The separation between the catheter hub and the needle hub can be performed without using the ultrasound probe. The separation can also be performed with just the holding hand. Optionally, the non-holding hand, after releasing the ultrasound probe and after successfully advancing the catheter tube further into the vein, can be used to hold the catheter hub steady, such as against the patient's skin, as the user retracts the needle hub with the holding hand.

As described, the catheter assembly of the present invention may be used in the same manner as a standard peripheral intravenous catheter (PIVC) assembly. Further, the steps of puncturing the patient, advancing the guidewire, advancing the catheter tube over the guidewire, and the step of retracting the needle can be performed using a one-handed procedure. The process does not require switching the holding hand used to hold the catheter assembly at the start of the procedure, or the use of both hands. Minimizing hand changing or handling locations of the catheter assembly can reduce undesired needle movement as the needle enters the vein and/or as the guidewire is dispensed into the vein to guide the catheter tube.

In another aspect of the invention, the needle hub can have a body with a nose section at a distal end and a tube guide at a proximal end. A working platform having an upper or working surface is provided between the distal end and the proximal end. A distal path or first path can be provided from a distal part of the working platform through the nose section and a proximal path or second path can be provided from a proximal part of the working platform through the tube guide at the proximal end of the catheter hub. In some examples, the working platform can embody other shapes than as shown. For example, the working surface of the working platform can be narrower, wider, longer, has curves, has side edges, etc.

A path opening or a perimeter defining a distal path opening can be provided as the opening of the distal path and a path opening or a perimeter defining a proximal path opening can be provided as the opening of the proximal path, both at the working platform. The two openings can be spaced from one another. The surface of the working platform can be located between the two openings, and wherein the surface can be exposed to the environment or atmosphere. The exposed surface between the two openings allows the guidewire passing between the two openings to be accessible by a user or practitioner.

By exposed, the working surface is not covered by any structure and access to the surface from a side of the needle hub body for pushing against by a user's finger is permitted or available. In some examples, the distance between the two openings is at least 1-inch, preferably at least 1.25-inches, and more preferably at least 1.5-inches, at least 2-inches, at least 2.5-inches, or at least 3-inches. The width of the needle hub is about 0.5-inch or greater, such as from about 0.75-inch to about 1.25-inches.

In alternative embodiments, the working platform can be covered, such as being overlaid by a transparent and pliable plastic sheet or plastic tube to cover the guidewire from blood splatter or exposure. The transparent plastic sheet can cover the working surface but is pliable or sufficiently soft to enable a user to press both the plastic sheet and the guidewire against the working surface to advance the guidewire. If a plastic tube is used, the plastic tube can provide a protective covering surrounding the guidewire and can conceal the guidewire. Both the transparent plastic sheet and plastic tube can be expandable and flexible, thus will not interfere the advancement or retract of guidewire during application.

In an example, the distal path opening and the proximal path opening are both generally round and generally of the same dimension. In other examples, the dimensions can differ and/or the shapes of the two openings can differ. For example, the proximal path opening, being an exit point for the guidewire, can be smaller in size than the distal path opening, which is an entry point when threading the guidewire from the proximal end, through the needle hub, and into the lumen of the needle.

The body of the needle hub can be viewed as having three distinct sections, including a first section, a second section, and a third section. The first section can include the nose section having a needle well for receiving and securing the needle proximal end thereto. The needle well can be located at an end of the distal path and the needle can attach to the needle well.

Exteriorly, the nose section can be sized and shaped to project into the catheter hub proximal end in a ready to use position. The nose section can have a Luer taper or can be a male stub sized to fit in the catheter hub. A flange or extension can be incorporated distal of the nose section. The extension can be sized and shaped for pushing a needle guard into the catheter hub during assembly. However, the extension may be omitted even if a needle guard is incorporated by using other tools or options.

The first section can further include a transition or neck section, which connects the nose section to the second section. The neck section can have a three-dimensional trapezoidal shape structure having a plurality of sides and having the distal path extending therethrough. The neck section can enlarge from a smaller distal end to a larger proximal end to bridge the size differences between the nose section and the second section. In other examples, the neck section can embody a different shape, such as having a frusto-conical shape. Further, while a nose section is shown at a distal end of the first section, the nose section may be designed out. For example, the first section can incorporate features that can grip or set the assembly position with the catheter hub for an external position, which as with an arm, a hood, a saddle, etc. that grips the catheter hub on the exterior of the catheter hub.

In an example, a receiving cavity is formed in the neck section. The receiving cavity can resemble a channel or a slot and is sized and shaped to accommodate a flow restrictor to restrict flow passing through the distal passage from the needle lumen. In an example, the flow restrictor can embody a septum, a valve, or a hydrophobic filter or hydrophobic membrane. In use and upon accessing the patient's vasculature, blood pressure causes blood to flow proximally through the needle lumen. When the flow restrictor is incorporated, proximal blood flow can be stopped or restricted at the flow restrictor, thereby eliminating or limiting blood leakage into the second section and onto the working platform.

In an example, the receiving cavity is sized and shaped to frictionally grip the flow restrictor or hold the flow restrictor in an interference fit. The receiving cavity can be a slot and part of the receiving cavity can bisect the distal path or the distal path can pass through or originates at the receiving cavity. In other examples, a retainer clip can be provided to snap over the opening of the receiving cavity to secure the flow restrictor therein. Preferably, the flow restrictor is sized and shaped to permit penetration by the guidewire and to permit movement of the guidewire therethrough during advancement of the guidewire.

The second section can be generally arc shape along a side view, in which the working surface of the working platform and the opposed bottom surface have a matching arc-shape. The two surfaces are separated from one another by a thickness that defines a thickness of the needle hub. The thickness can be constant or can vary or non-constant along the length of the working platform. In other examples, the two surfaces can have different arcs. In still yet other examples, the bottom surface can be flat or generally planar or can have a combination of planar and undulating surfaces.

The surface of the working platform is preferably arc-shape. The arc-shape surface helps to move the working surface of the working platform closer to the practitioner's finger as the practitioner uses his or her finger to slide the guidewire against the surface from a proximal position to a distal position. However, the surface is not limited to an arc shape. For example, the working surface of the working platform can be generally planar or can have different degrees of arc-shape. Thus, where the second section can have two upper reference points defining a plane, the surface of the working platform can locate below the or subjacent the plane and can have an arc-shape.

In addition to the upper surface and the bottom surface, the second section has two opposed sides. The sides can be smooth or flat or can have surface features, as previously discussed. The sides can alternatively have grooves to receive fingers of the holding hand.

The third section of the needle hub can optionally include a guide stub. The guide stub can be unitarily formed, such as by single plastic injection molding, with the first and second sections or can be separately formed and secured to the first and second sections. For example, the guide stub can be separately molded with a bore or passage and then glued, snap-fit, or welded to the proximal end surface of the second section. If separately formed, the part of the proximal path formed with the second and the part of the proximal path formed with the guide stub should align.

In an example, the guide stub is generally cylindrical. In other examples, the guide stub can have multi-sides, such as having a polygonal shape. The guide stub extends the overall length of the proximal path and can provide an anchor point or attachment point for a tubing, as previously discussed. However, the guide stub may be omitted and the second section can be provided with a sufficient path length and serves as an attachment point for the tubing. For example, from the end point of the second section, the second section can be extended in the proximal direction so that the proximal end surface is extended further proximally instead of coinciding with the end point. This allows the path to be lengthened and the tubing can couple directly to the end opening thereof.

In yet another aspect of the invention, the needle hub has a body with a neck section that has been modified to have a generally cylindrical shape and the receiving cavity has been modified to receive a relatively smaller flow restrictor. The flow restrictor can be located at the distal path opening of the distal path and gripped along at least two sides, preferably at least three sides, by the structure of the receiving cavity. Optionally, the flow restrictor can be retained to the receiving cavity, such as with a clip, a detent engagement, by adhesive, or combinations thereof.

The second section of the needle hub comprises a working platform having a surface and a lower surface, which can both be arc-shaped. In the present embodiment, the working platform resembles a bowl shape channel and the upper or working surface of the working platform can be located at the bottom of the channel. The working platform can have two upwardly extending sides that extend from the working surface. Along an end cross-section, the bowl shape channel resembles the letter “U”, but with relatively lower extending sidewalls resembling a short U. Further, the channel arcs or curves as the surface extends from the proximal end to the distal end. For example, the working surface and the upper edge of both sidewalls have an arc-shape. The channel helps to locate the practitioner's finger between the two sidewalls when using the catheter assembly to advance the guidewire.

In an example, the guide stub of the third section can be a separately formed component that is attached to the second section of the body, such as by adhesive or welding. However, the guide stub be singularly formed with the second section or can be omitted altogether. If separately formed, then the first proximal path section of the proximal path can align with the second proximal path section of the proximal path to define an overall proximal path. As shown, the upper contour of the guide stub can be generally flat or flush with the upper proximal point of the second section. In other examples, the upper contour of the guide stub can locate above or below with the upper proximal point of the second section.

The rear-most opening of the proximal path is preferably enlarged or is preferably larger than the diameter of the path itself. The rear-most opening can be enlarged to facilitate insertion of the guidewire. The rear-most opening can be enlarged to receiving a tubing. The tubing can attach to the rear-most opening via friction fit, interference fit, by welding, by adhesive, or combinations thereof.

In use, a user can grip the catheter assembly with one hand in a supinated position. The catheter assembly can comprise a catheter unit and a needle unit. The catheter unit can embody any of the catheter units described elsewhere herein. The user can hold the catheter assembly with the holding hand, which can be a left hand or a right hand. The user can puncture the patient at a puncture site and the needle tip and catheter tube opening can enter the vein, which causes blood to flow upward through the lumen of the needle.

In an example, proximal blood flow through the needle is stopped or restricted by a flow restrictor. The flow restrictor can be located in a receiving cavity formed with the needle hub. The flow restrictor, which can be a septum, a valve, or a hydrophobic filter, can restrict or limit blood flow from flowing onto and wetting the surface of the working platform.

The user can advance a guidewire distally out the needle tip and into the vasculature of the patient. When the guidewire is advanced distally, the guidewire will at least move through the flow restrictor. Because a flow restrictor can be incorporated with the integrated guidewire and needle hub, blood flowing through the needle lumen can be restricted or blocked from spilling onto the working platform.

Methods of making and of using the catheter assemblies and components thereof are within the scope of the present invention.

### BRIEF DESCRIPTION OF THE DRAWINGS

These and other features and advantages of the present devices, systems, and methods will become appreciated as the same becomes better understood with reference to the specification, claims and appended drawings wherein:

FIG. 1 is a perspective view of a catheter assembly provided in accordance with aspects of the present invention, which comprises a catheter unit and a needle unit having a needle and a guidewire.

FIG. 2 is a schematic perspective view of a practitioner holding a catheter assembly with a supported tubing.

FIG. 3 is a schematic perspective view of a practitioner holding a catheter assembly with an unsupported tubing.

FIG. 4A is a perspective view of a catheter assembly provided in accordance with further aspects of the invention, which comprises a catheter unit and a needle unit having a needle and a guidewire.

FIG. 4B is a partial cross-sectional side view of the catheter unit of FIG. 4A.

FIGS. 5(A)-5(C) show a sequence of views of a user using a catheter assembly of the invention and dispensing the guidewire with the same holding hand.

FIG. 6 is a perspective view of a catheter assembly with an unsupported tubing.

FIG. 6A is an enlarged view of the proximal end of the guidewire.

FIGS. 7(A)-7(D) show a sequence of views of a user using a catheter assembly of the invention and dispensing a guidewire with the same holding hand and holding an ultrasound probe in the other hand.

FIGS. 8A and 8B show a perspective view and a side view, respectively, of a needle unit having a flow restrictor.

FIG. 9 shows a cross-sectional side view of a needle unit in accordance with further aspects of the invention having a flow restrictor.

FIG. 10 is a schematic view of a practitioner holding a catheter assembly having a flow restrictor.

FIG. 11 is a schematic view of a practitioner holding a catheter assembly having a flow restrictor and dispensing a guidewire with the same holding hand.

### DETAILED DESCRIPTION

The detailed description set forth below in connection with the appended drawings is intended as a description of the presently preferred embodiments of catheter assemblies and components thereof provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

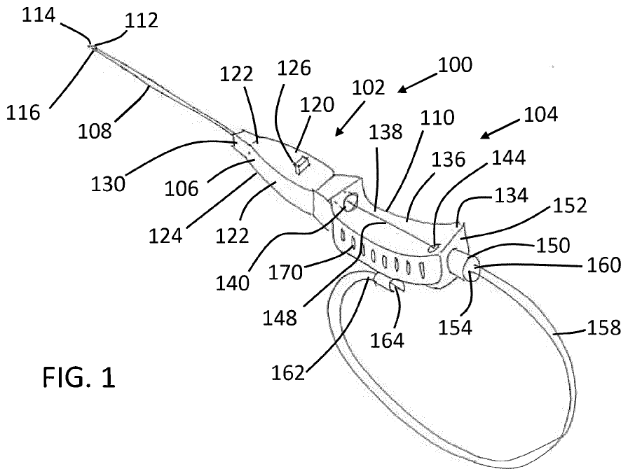

With reference now to FIG. 1, a catheter assembly 100 in accordance with aspects of the invention is shown comprising a catheter unit 102 and a needle unit 104. In an example, the catheter unit 102 comprises a catheter hub 106 and a catheter tube 108 extending from the catheter hub 106. The needle unit 104 comprises a needle hub 110 having a needle 112 attached thereto and the needle extending in a distal direction through the catheter hub 106 and the lumen of the catheter tube 108 with the needle tip 114 projecting distally of the distal opening 116 of the catheter tube in a ready to use position.

The catheter hub 106 can have a hub body 120 having a proximal opening with a female Luer receiving a distal end of the needle hub 110. The hub body 120 can have a plurality of sides 122 connected to one another along connecting corners 124 or can have a traditional frusto-conical body, as further discussed below. A push tab 126 can locate on the upper portion of the hub body 120 for use by a practitioner to push on to separate the catheter hub from the needle hub following successful venipuncture. A nose section 130 can locate at the distal end of the hub body 120 and can have a smaller cross-sectional dimension than the proximal portion of the hub body 120, such as by providing one or more tapered surfaces between the proximal end and the distal end of the hub body 120. The hub body may be made from traditional plastic materials used for catheter hubs with a translucent appearance.

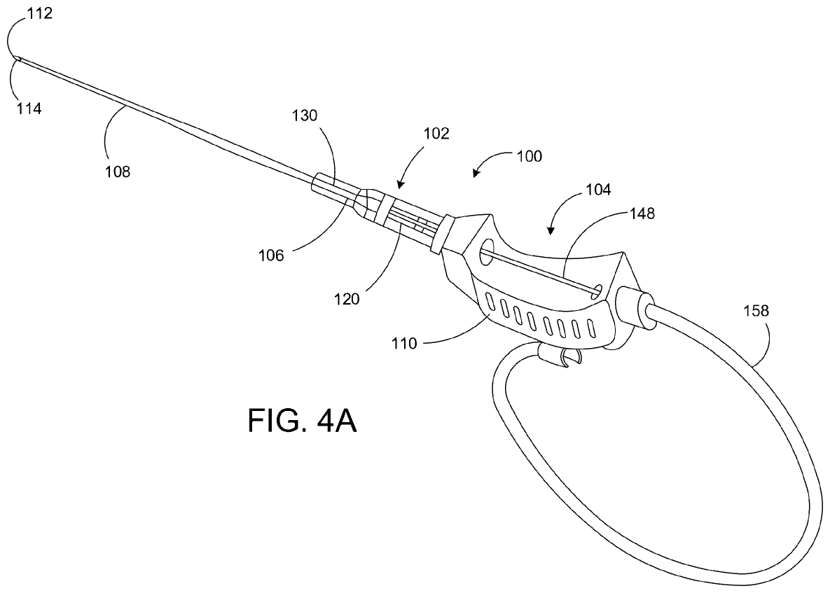

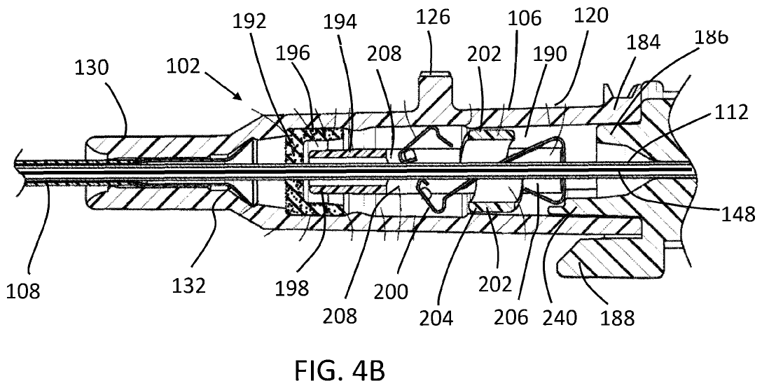

In an example, the needle hub 110 can comprise a body 134 having a length and a width. The body 134 can have a generally polygonal shaped cross-section with a plurality of sides that include a working platform 136, an irregular shaped cross-section with a working platform 136, or a generally circular shaped cross-section with a working platform 136. The working platform 136 can be defined by an upper surface or working surface 138 that a user or practitioner of the catheter assembly 100 can push with at least one of his or her fingers, such as the index finger, against to dispense a guidewire, as further discussed below. The needle hub 110 can have a nose section that projects into the interior of the catheter hub 106 in the ready to use position. The catheter hub 106 can have a female Luer at the proximal end for receiving the nose section of the needle hub. The catheter hub 106 can also optionally incorporate a needle guard, a valve and/or a valve opener in the interior of the hub body 120, such as shown in FIG. 4B.

In an example, the body 134 of the needle hub 110 comprises a distal path 140 and a proximal path 144. The two paths 140, 144, can be arranged or aligned to pass a guidewire 148 therethrough or therebetween. From the working platform 136, the guidewire 148 can have a length that extends in the distal direction, through the distal path 140, through the lumen of the needle 112, and having a guidewire distal end that terminates proximal of the needle tip opening. From the working platform 136, the guidewire 148 can have a length that extends in the proximal direction and out through the proximal path 144 of the needle hub 110. The needle hub 110 can incorporate a guide stub 150 extending from the proximal end surface 152 of the body 134 to extend the length of the proximal path. The guide stub 150, when incorporated, has a bore 154 aligned and in communication with the proximal path 144 that originates at the working platform 136. The guide stub 150 can be omitted from the body 134 and an opening can be provided at the proximal end surface 152 of the body 134 for the guidewire to exit in the proximal direction.

As shown, a tubing 158 having a length with a first end or distal end 160 engages the opening or bore 154 of the guide stub 150, such as in an interference fit. Optionally, adhesive or glue may be used to more permanently secure the distal end 160 in the bore 154. The tubing 158 can be flexible and be made from any number of prior art tubing materials. The tubing 158 can be hollow or has a lumen and provided to store or accommodate the proximal part of the guidewire 148 therein, the part that extends in the proximal direction out through the proximal path 144. The proximal end 162 of the tubing 158 can be supported. For example, the proximal end 162 can connect to a socket 164 on the needle hub body 134, which can be an open socket with a gap or a closed socket without a gap. By engaging the socket 164, the tubing 158 can coil into a loop or a partial loop to organize the tubing and the guidewire located therein, such as to keep the tubing and the guidewire from dangling out the proximal end of the needle hub 110. In other examples, the tubing 158 can be unsupported at the proximal end of the tubing. For example, the tubing can extend from the needle hub 110 without engaging the needle hub at the second end of the tubing.

As shown, the guidewire 148 has a length and a proximal end that is located inside the lumen of the tubing 158. How far or long does the guidewire 148 extend into the tubing 158 can depend on how much of the guidewire is permitted to be dispensed out the needle tip 114, as further discussed below. In an example, the overall length of the guidewire 148 is selected so that upon being dispensed by the user, the proximal end of the guidewire terminates within the working platform 136, which signifies the end of the guidewire length and therefore the guidewire cannot physically be dispensed any further. In other examples, the guidewire can be provided with an enlarged section, such as an integrated notch, bump, build-up, a J-loop proximal end, or a sleeve, and upon dispensing the guidewire, the enlarged section is physically stopped by a relatively smaller opening formed with the needle hub 110, such as with the guide stub 150, the body 134, the distal path 140, or the proximal path 144. In still other examples, the needle 112 can have a crimp and wherein distal advancement of the guidewire is stopped when the enlarged section of the guidewire contacts the crimp from inside the needle lumen.

As further discussed below, the guidewire 148 is slidable or movable within the tubing 158 when using a finger, such an index finger, to press the guidewire against the surface 138 of the working platform 136 and simultaneously sliding the guidewire against the surface 138 in the distal direction to dispense the distal end of the guidewire 148 out the needle tip. As the needle unit 104 carries both the needle 112 and the guidewire 148 without a separate guidewire dispenser having a guidewire dispenser housing, the needle unit 104 can be referred to as an integrated needle and guidewire hub and the catheter assembly 100 can be referred to as a catheter assembly with an integrated needle and guidewire hub.

In an example, a plurality of spaced apart surface features 170 can be provided on one or more sides of the needle hub 110. The space apart surface features 170 can embody bumps, recesses, or a combination of bumps and recesses of any number of shapes. As shown, the spaced apart surface features 170 are bumps having an oblong shape or an oval shape with other shapes contemplated, such as a round shape, a square shape, a star shape, an irregular shape, etc. The surface features 170 can improve gripping or can simply serve as aesthetic features.

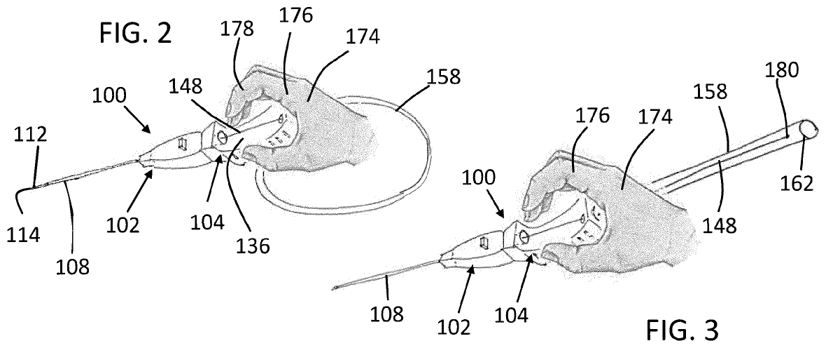

FIG. 2 is a schematic view showing a practitioner or user 174, such as a doctor or a nurse, holding the catheter assembly 100 using a one-handed technique. In particular, the hand 176 grips the needle unit 104 in a supinated position with the index finger 178 located above or over the working platform 136 to advance the guidewire 148 following vascular access with the needle tip 114. The figure shows the hand being a right-hand, which is exemplary only as the user can optionally use the catheter assembly 100 with a left-hand.

The tubing 158, as more clearly shown in FIG. 1, is coiled below the hand 176 with the first end or distal end 160 coupled to the guide stub 150 and the second end or proximal end 162 of the tubing supported by a socket 164. A section of the guidewire 148, including the proximal end of the guidewire, is located inside the lumen of the tubing 158 and freely slidable inside the tubing when advanced by the user 174.

FIG. 3 is a schematic view showing a user 174 holding the catheter assembly 100 using a one-handed technique, similar to that of FIG. 2. In the present embodiment, the first end of the tubing 158 is shown attached to the needle unit 104 but wherein the second end 162 is spaced or not attached to the needle unit 104, such as being unsupported. The tubing 148 may be generally straight as shown by incorporating a thicker, stiffer or a relatively higher durometer tubing material, or the tubing can bend or curved as it extends from the needle unit 104 by incorporating a thinner, softer, or relatively lower durometer tubing material.

FIG. 3 also shows a guidewire 148 extending into the lumen of the tubing 158 and showing the second end or proximal end 180 of the guidewire located inside the tubing lumen but extending short of the proximal opening at the second end 162 of the tubing. The guidewire may be of a peripheral type, typically with a core diameter of about 0.014-in, 0.018-in, or 0.035-inch, to name a few non-limiting examples, and can be made from any number of prior art materials, such as stainless steel, nitinol, or a hybrid type.

FIG. 4A shows a catheter assembly 100 provided in accordance with further aspects of the invention. The present catheter assembly 100, like the catheter assembly of FIG. 1, comprises a catheter unit 102 and a needle unit 104. The needle unit 104 can be similar to the needle unit 104 of FIG. 1. The catheter unit 102 can have a catheter hub 106 and a catheter tube 108 extending therefrom. The present catheter hub 106 embodies a more traditional elongated generally round, generally frustoconical, hub body 120. As shown, the catheter hub 106 comprises a nose section 130 having the catheter tube 108 extending therefrom and a proximal opening receiving the nose section of the needle hub 110 in the ready to use position. Internally of the catheter hub 106, a needle guard for covering the needle tip 114 and/or a combination of a valve and valve opener can be positioned for controlling fluid flow through the catheter hub, as further discussed below with reference to FIG. 4B.

FIG. 4B is a partial cross-sectional side view of the catheter unit 102 of FIG. 4A, which has a body 120 with a proximal opening 184 having a female Luer having the nose section 186 of the needle hub 110 located therein. An optional tab or flange 188 can be provided adjacent the nose section 186 to grip part of the catheter hub 106 therebetween. The proximal opening 184 of the catheter hub 106 has exterior threads and may be referred to as a female threaded Luer.

The catheter hub 106 has a body 120 defining an interior cavity 190. The catheter tube 108 extends distally of the nose section 130 of the catheter hub and is held to the catheter hub by a metal bushing or a ferrule 132. A valve or septum 192 is located in the interior cavity 190 between a distal shoulder and a proximal shoulder formed on the interior surface of the catheter hub to retain the valve 192 therebetween. The valve can have a plurality of slits defining a plurality of flaps. The flaps can open to permit flow through the catheter hub or close to restrict or limit flow through the catheter hub. A valve opener or septum opener 194, also referred to as an actuator, having a nose section 198 is located proximally of the valve 192 and inside a space defined by the skirt 196 of the valve. The nose section 198 of the valve opener 194 is generally cylindrical and has a bore having the needle 112 passing therethrough, which has the guidewire 148 located in the lumen thereof. One or more openings can be provided through the body of the nose section 198 for fluid flushing.

The valve opener 194 has a plunger element 206 extending proximally from the nose section 198. In an example, two plunger elements 206, 206 extend from the nose section 198 and having a gap therebetween for accommodating a needle guard 200, which can be located in the gap between the two plunger elements 206, 206. A pair of bands or stabilizers 202, 202 can be provided with the valve opener 194. In an example, each band or stabilizer 202 connects to both of the two plunger elements 152, 152 so that a continuous ring or loop 204 is formed at the two bands 202, 202 and the two plunger elements 206, 206. The ring or loop 204 is spaced from the nose section 198. Accordingly, through holes or through openings 208, 208 are provided with the valve opener 194 between the nose section 198 and the ring 204. In an example, at least two through holes are present between the nose section and the ring 204.

In an example, the needle guard 200 has a proximal wall with a perimeter defining an opening for accommodating the needle 112 passing through the proximal wall. A pair of arms extend distally of the proximal wall with each arm having a distal wall and a curved lip at an end thereof. An elbow is defined between the arm and the distal wall of each arm and the two elbows define a maximum guard dimension when the two arms are biased outwardly by the needle in the ready to use position as shown in FIG. 4B. The needle guard 200 is positioned at the gap between the two plunger elements 206, 206 with the proximal wall located proximally of the ring 204 and the two elbows located distally of the ring, in the two through openings 208, 208 of the valve opener. In an example, the two elbows can contact the interior of the catheter hub 106, can be spaced from the interior of the catheter hub, can contact one or both of the two bands or stabilizers 202, 202, can be spaced from the two bands, or combinations thereof in a ready to use position.

In use, when the needle 112 retracts in the proximal direction following successful venipuncture and the needle tip moves proximally of the two distal walls of the needle guard 200 and the two arms no longer biased outwardly by the needle, the two arms can recoil or move inwardly so that the profile at the two elbows is now smaller than the inside diameter of the ring 204, and a crimp on the needle (not shown) can abut or contact the perimeter defining the opening on the proximal wall to then pull the needle guard 200 out of the catheter hub 106 along with the needle 112.

The valve 192 and the valve opener 194 can remain with the catheter hub 106 to control fluid flow through the catheter hub. For example, a male Luer tip, such as a syringe tip, can be inserted into the female Luer to advance the valve opener 194 distally to then open the valve with the nose section 198 of the valve opener. The valve 192 can have a plurality of slits defining a plurality of flaps that deflect by the nose section of the valve opener when the valve opener is advanced distally. In an example, the valve has three slits defining three flaps. The needle guard and the combination valve and valve opener, or both, can be incorporated with other catheter hubs, such as the catheter hub 106 of FIGS. 1-3.

Further aspects of the catheter unit 102, such as the valve 192, the valve opener 194, and the needle guard 200, are disclosed in U.S. Pub. No. 2018/0214682, the contents of which are expressly incorporated herein by reference for all the publication discloses.

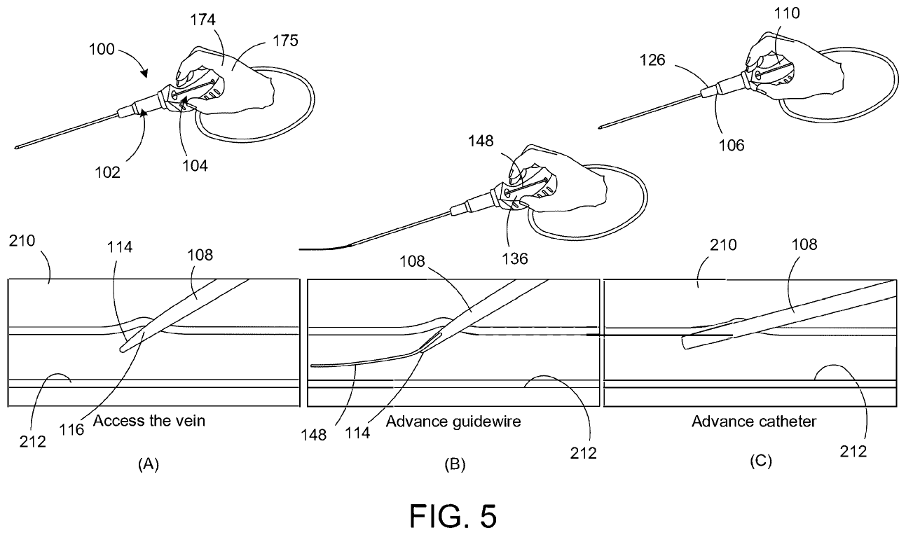

FIG. 5 shows a sequence of views explaining a one-handed use capability of the catheter assembly 100 of the present invention. Starting with FIG. 5(A), a user can grip the catheter assembly 100 with one hand in a supinated position as previously discussed. The hand used to grip and hold the catheter assembly may be referred to as the holding hand 175, which can be a left hand or a right hand. The user can then poke the patient 210 to access the vein 212 with the needle tip 114 and catheter tube opening 116 of the catheter tube 108. Proper needle puncture can be confirmed via primary blood flashback. Alternatively or additionally, an ultrasound probe or meter, such as a 2D or 3D ultrasound probe, may be used to verify proper needle puncture. If used, the ultrasound probe may be held with the free hand, the one without the catheter assembly. In some examples, the needle 112 used can be selected to have the desired stiffness to minimize bending, particularly if the needle is longer than a standard peripheral intravenous catheter (PIVC). A sufficiently sized gauge needle, a ribbed needle, a dual diameter needle, or a multi-diameter needle may be used.

FIG. 5(B) shows the user advancing the guidewire 148 while steadily holding the catheter assembly 100 in the same holding hand 175 roughly in the same position to limit or avoid moving the needle tip inside the vein 212 from the targeted area. The user can advance the guidewire 148 by using one of the fingers, such as the index finger, on the same hand that is holding the catheter assembly 100 to press the guidewire against the surface 138 of the working platform 136 (FIG. 1) and then using friction between the fingertip and the surface 138 incrementally advancing the wire by moving the finger between a proximal position on the working platform 136 to a distal position, and then lifting the finger back to the proximal position on the working platform and repeat. The guidewire 148 can advance until a physical stop is activated, felt or triggered, such as when a crimp, a bump, or an enlarged section on the guidewire abuts the crimp on the needle from the inside of the needle, until the end of the guidewire is reached and no further guidewire is available for advancing, until the enlarged section on the guidewire abuts a narrow opening on the needle hub, or combinations thereof.

After the guidewire 148 is advanced into the vein 212, the catheter tube 108 is then slid or advanced over the guidewire and the guidewire 148 serves to support and guides the catheter tube into the vein 212. In an example, the catheter tube 108 can advance over the guidewire 148 by using one of the fingers of the holding hand to push the push tab 126 on the catheter hub 106 in the distal direction, as shown in FIG. 5(C). This action causes the catheter hub 106 to separate from the needle hub 110. The separation can continue until the needle 112, the guidewire 148, and the needle hub 110 are completely separated from the catheter hub 106 and the tip of the catheter tube 108 remaining inside the vein for vascular access. Thus, in moving from FIG. 5(A) to FIG. 5(C), the user can use the same holding hand to manipulate the catheter assembly without having to change hand or to use both hands.

With reference again to FIG. 4B in combination with FIG. 5(C), the catheter unit 102 can optionally have a needle guard 200 for covering the needle tip 114 in a protective position of the needle. In addition to or alternatively, the catheter unit 104 can have a valve or septum 192 and a valve opener 194. When incorporated, the distal walls of the needle guard 200 can each have a vertical slit sized to accommodate the diameter of the guidewire 148 but not the needle. The slits allow the distal walls to move distal of the needle tip in the protective position and accommodate the guidewire 148 that extends out of the needle as the needle is withdrawn following successful venipuncture. When incorporated, the valve or septum 192 and the valve or septum opener 194 remain with the catheter hub 106 for controlling flow through the catheter hub.

As described, the catheter assembly 100 of the present invention may be used in the same manner as a standard peripheral intravenous catheter (PIVC) assembly. That is, the initial puncturing of the patient and the separation of the needle and needle hub from the catheter hub following successful venipuncture involve essentially the same techniques, which can be performed using a one-handed technique. Additionally, the step of advancing the guidewire does not require switching the holding hand to the other hand, using both hands, or separating the holding hand from the needle hub. Minimizing hand changing or handling locations of the catheter assembly can reduce undesirable needle movement as the needle enters the vein and/or as the guidewire is advanced into the vein to guide the catheter tube. These benefits provide for easy adaptation and acceptance by practitioners of the disclosed catheter assemblies.

As shown and described, the catheter assemblies with integrated guidewire dispensing needle hub of the present invention can have at least one less attachment point normally found between the needle hub and a guidewire dispenser housing. Thus, as prior art needle hubs and guidewire dispenser housings can be provided in separate packages, the present catheter assembly can reduce at least one pre-procedure assembly step. The present catheter assembly with integrated guidewire dispensing needle hub can also be practiced without an outer removable or separable housing used to guide the catheter hub during separation of the catheter hub from the needle hub typically found in midline catheters.

Still further, the present catheter assembly design, including the disclosed needle unit with an integrated guidewire and needle hub, can be used without a guidewire and used as a standard peripheral intravenous catheter (PIVC) assembly. For example, the catheter assembly as shown and described can be used without a guidewire.

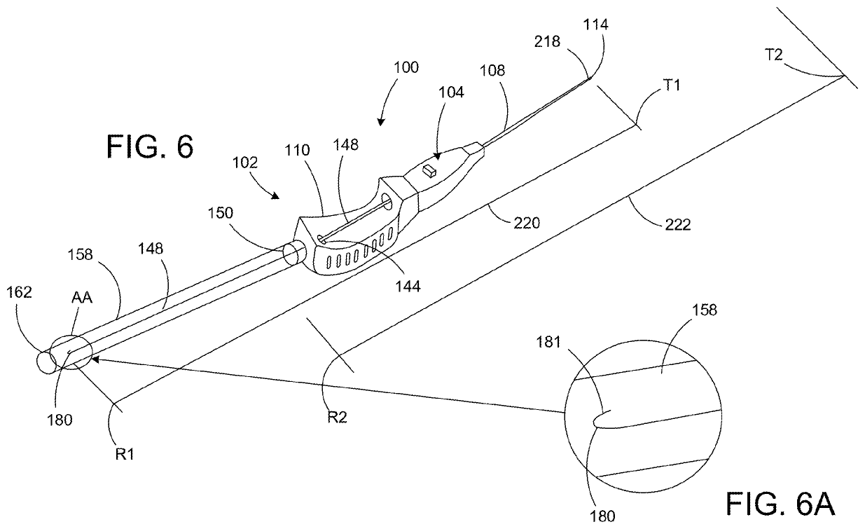

FIG. 6 is a schematic view showing the catheter assembly 100 of the present invention with the tubing 158 length extending out from the needle hub 110 and unsupported to show movement of the guidewire 148 when in use. The same concept applies when the tubing 158 is supported at the proximal end 180 of the tubing. The same concept applies equally when the catheter unit 104 has a generally cylindrical, generally frusto-conical, shaped body as shown in FIGS. 4A and 4B.

The guidewire positioned in the ready to use position is represented by line 220, which shows the distal tip or guidewire tip 218 of the guidewire recessed proximally from the needle tip 114 of the needle and the proximal end 180 of the guidewire 148 distal of the proximal end opening 162 of the tubing 158. The guidewire tip 218 is located at point T1 within the needle 112 and the proximal end 180 of the guidewire 148 is located within the tubing 158 at point R1. Said differently, the distal tip 218 of the guidewire is recessed proximally from the needle tip 114 at point T1 and the rear 180 of the guidewire 148 is recessed from the proximal end opening 162 of the tubing 158 at point R1.

In use, the guidewire 148 can be dispensed and pushed distally out through the needle tip 114 as previously discussed. In an example and with reference to line 222, the distal tip 218 of the guidewire can be dispensed distally of the needle tip 114 to point T2 and the proximal end 180 of the guidewire 148 can move distally to point R2. The distance between the needle tip 114 and point R2, call the guidewire dispensed length, can be selected to provide adequate guide and support for the catheter tube 108 to advance the catheter tube 108 into the targeted vein for vascular access.

With reference to FIG. 6A, which is an expanded view of point AA of FIG. 6, the guidewire proximal end 180 can be coiled to increase the overall size or profile of the guidewire at the proximal end to define an enlarged end 181. The enlarged end 181 having an enlarged profile relative to the guidewire diameter at the proximal end 180 can be used to engage or be trapped by the needle hub, such as by a small opening in the guide stub 150 if incorporated or by a small opening in the main body 134 of the needle hub 110. In an example, the enlarged end 181 can be a J-shaped tip or a coiled tip. The needle hub 110, such as the guide stub 150 or the proximal path 144, can have a bore for accommodating the diameter of the guidewire 148 but too small for the enlarged end 181 of the guidewire to pass therethrough. Thus, when the guidewire 148 is dispensed during use, engagement between the enlarged end 181 and the structure defining the bore can stop further distal advancement of the guidewire 148, which correspondingly stops distal advancement of the guidewire tip 218 further distally relative to the needle tip 114 and therefore defines the dispensed length of the guidewire.

In alternative embodiments, the guidewire 148 can have a bump or an enlarged section, such a material build-up, that is larger than the internal path through the lumen of the needle 112 at the crimp section of the needle, near the needle tip 114. Upon being dispensed, the bump or build-up on the guidewire can engage the crimp on the needle to stop distal advancement of the guidewire. The crimp on the needle can thus be used to both engage and retract a needle guard and stop further distal advancement of the guidewire.

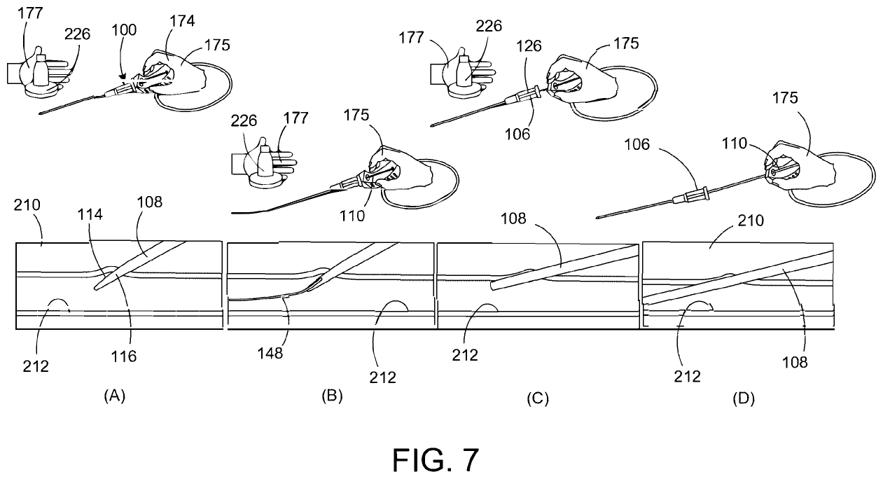

FIG. 7 shows a sequence of views explaining the one-handed use capability of the catheter assembly of the present invention used in combination with an ultrasound probe, such as a 2D or 3D ultrasound probe. Starting with FIG. 7(A), a user 174 can grip the catheter assembly 100 with one hand in a supinated position as previously discussed. The user can hold the catheter assembly 100 with the holding hand 175, which can be a left hand or a right hand, and an ultrasound probe in the other hand, referred to as the probe hand 177 for holding an ultrasound probe 226. The user can then poke the patient 210 using the holding hand 175 to access the vein 212 with the needle tip 114 and the catheter tube opening 116. The user can use the ultrasound probe 226 with the probe hand 177 to monitor the puncture site for proper placement of the needle tip 114. Optionally, proper needle puncture can be confirmed via primary blood flashback in addition to verification with the ultrasound probe 226.

FIG. 7(B) shows the user advancing the guidewire 148 out the needle while steadily holding the catheter assembly 100 in the same holding hand 175 and roughly in the same position to limit or avoid moving the needle tip inside the vein 212 from the targeted area. The user can advance the guidewire 148 by using one of the fingers, such as the index finger, on the same holding hand 175 that is holding the catheter assembly 100 to press the guidewire against the surface 138 of the working platform 136 (FIG. 1) and then using friction between the fingertip and the surface 138 to incrementally advance the wire by moving the finger between a proximal position on the working platform 136 to a distal position, and then lifting the finger back to the proximal position on the working platform and repeat. The guidewire 148 can advance under review or monitor of the ultrasound probe 226 held in the probe hand 177 until physically stopped by an engagement between the guidewire 148 and the needle hub 110 or between the guidewire and the crimp on the needle, as previously discussed.

After the guidewire 148 is advanced into the vein 212, the catheter tube 108 is then slid over the guidewire and the guidewire 148 serves to support and guides the catheter tube into the vein, as shown in FIG. 7(C). In an example, the catheter tube 108 can advance over the guidewire 148 by using one of the fingers of the holding hand 175 to push the push tab 126 on the catheter hub 106 in the distal direction. This action causes the catheter hub 106 to separate from the needle hub 110. Further catheter tube advancement can be monitored by the ultrasound probe 116 held in the probe hand 177. Thus, in moving from FIG. 7(A) to FIG. 7(C), the user can use the same holding hand to manipulate the catheter assembly without having to change hand or to use both hands.

The user can continue separating the needle 112, the guidewire 148, and the needle hub 110 by moving the needle hub and the catheter hub away from one another until the needle hub, needle, and guidewire are completely separated from the catheter hub 106, as shown in FIG. 7(D).

After complete separation, the tip of the catheter tube 108 remains inside the patient's vein for vascular access. The separation shown in FIG. 7(D) can be performed without using the ultrasound probe 226. The separation can also be performed with just the holding hand 175. Optionally, the non-holding hand 177, after releasing the ultrasound probe and after successfully advancing the catheter tube further into the vein, can be used to hold the catheter hub 106 steady, such as against the patient's skin, as the user retracts the needle hub 110 with the holding hand 175.

As described, the catheter assembly 100 of the present invention may be used in the same manner as a standard peripheral intravenous catheter (PIVC) assembly. Further, the steps of puncturing the patient, advancing the guidewire, advancing the catheter tube over the guidewire, and the step of retracting the needle can be performed using a one-handed procedure. The process does not require switching the holding hand 175 used to hold the catheter assembly at the start of the procedure, or the use of both hands. Minimizing hand changing or handling locations of the catheter assembly can reduce undesired needle movement as the needle enters the vein and/or as the guidewire is dispensed into the vein to guide the catheter tube.

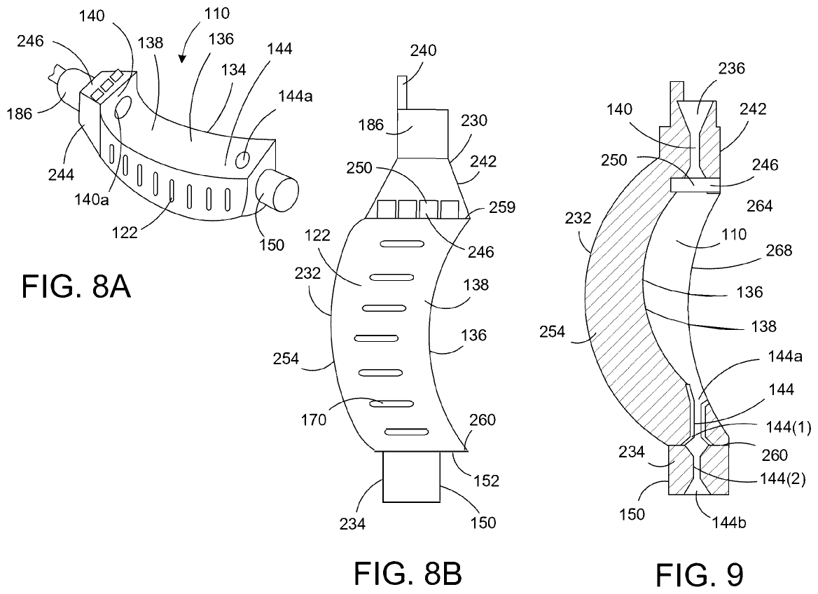

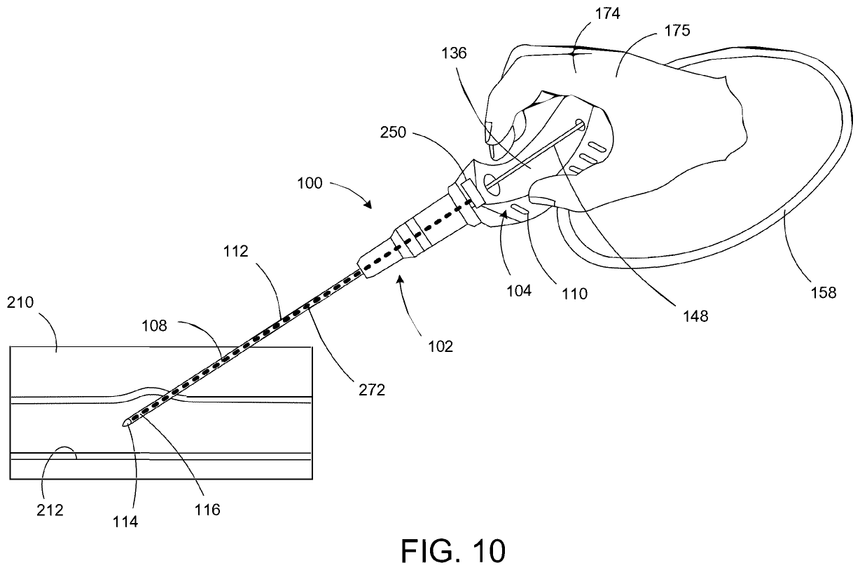

FIG. 8A is a perspective view of a needle hub 110 shown without a needle. FIG. 8B is a side view of the needle hub 110 of FIG. 8A. FIG. 9 is a cross-sectional side view of a needle hub 110 in accordance with further aspects of the invention, which slightly modifies the needle hub of FIG. 8B. Refer initially to FIG. 8A, the needle hub 110 is similar to the needle hub described elsewhere herein, such as that of FIGS. 1, 2, and 3 with a few differences. The present embodiment comprises a hub body 134 having a nose section 186 at a distal end and a tube guide 150 at a proximal end. A working platform 136 having an upper or working surface 138 is provided between the distal end and the proximal end. A distal path or first path 140 is provided from a distal part of the working platform 136 through the nose section 186 and a proximal path or second path 144 is provided from a proximal part of the working platform through the tube guide 150 at the proximal end of the catheter hub 110. In some examples, the working platform 136 can embody other shapes than as shown. For example, the working surface of the working platform can be narrower, wider, longer, has curves, has side edges, etc.

A path opening or a perimeter defining a distal path opening 140a is provided as the opening of the distal path 140 and a path opening or a perimeter defining a proximal path opening 144a is provided as the opening of the proximal path 144, both at the working platform. The two openings 140a, 144a are spaced from one another. The surface 138 of the working platform 134 is located between the two openings 140a, 144a, and wherein the surface 138 is exposed to the environment or atmosphere. The exposed surface 138 between the two openings 140a, 144a allows the guidewire 148 passing between the two openings to be accessible by a user or practitioner, as previously discussed. Thus, by exposed, the working surface 138 is not covered by any structure and access to the surface from a side of the needle hub body for pushing against by a user's finger is permitted or available. In some examples, the distance between the two openings is at least 1-inch, preferably at least 1.25-inches, and more preferably at least 1.5-inches, at least 2-inches, at least 2.5-inches, or at least 3-inches. The width of the needle hub is about 0.5-inch or greater, such as from about 0.75-inch to about 1.25-inches. In alternative embodiments, the working platform can be covered, such as being overlaid, by a transparent and pliable plastic sheet or plastic tube to cover the guidewire from blood splatter or exposure. The transparent plastic sheet can cover the working surface but is pliable or sufficiently soft to enable a user to press both the plastic sheet and the guidewire against the working surface to advance the guidewire. If a plastic tube is used, the plastic tube can provide a protective covering surrounding the guidewire and can conceal the guidewire. Both the transparent plastic sheet and plastic tube can be expandable and flexible, thus will not interfere the advancement or retract of guidewire during application.

In an example, the distal path opening 140a and the proximal path opening 144a are both generally round and generally of the same dimension. In other examples, the dimensions can differ and/or the shapes of the two openings can differ. For example, the proximal path opening 144a, being an exit point for the guidewire, can be smaller in size than the distal path opening 140a, which is an entry point when threading the guidewire 148 from the proximal end, through the needle hub 110, and into the lumen of the needle 112.

With further reference to FIG. 8B, the body 134 of the needle hub 110 can be viewed as having three distinct sections, including a first section 230, a second section 232, and a third section 234. The first section 230 includes the nose section 186 having a needle well 236 (FIG. 9) for receiving and securing the needle proximal end thereto. The needle well 236 can be located at an end of the distal path 140 and the needle can attach to the needle well. Exteriorly, the nose section 186 can be sized and shaped to project into the catheter hub proximal end in a ready to use position, as shown in FIGS. 1, 2, 3, and 4A, among others. The nose section 186 can have a Luer taper or can be a male stub sized to fit in the catheter hub. A flange or extension 240 can be incorporated distal of the nose section 186. The extension 240 can be sized and shaped for pushing a needle guard into the catheter hub during assembly, as shown in FIG. 4B. However, the extension 240 may be omitted even if a needle guard is incorporated by using other tools or options.

The first section 230 can further include a transition or neck section 242, which connects the nose section 186 to the second section 232. The neck section 242 can have a three-dimensional trapezoidal shape structure having a plurality of sides 244 and having the distal path 140 extending therethrough. The neck section 242 enlarges from a smaller distal end to a larger proximal end to bridge the size differences between the nose section 186 and the second section 232. In other examples, the neck section 242 can embody a different shape, such as having a frusto-conical shape. Further, while a nose section 186 is shown at a distal end of the first section 230, the nose section may be designed out. For example, the first section 230 can incorporate features that can grip or set the assembly position with the catheter hub for an external position, which as with an arm, a hood, a saddle, etc. that grips the catheter hub on the exterior of the catheter hub.

In an example, a receiving cavity 246 is formed in the neck section 242. The receiving cavity 246 can resemble a channel or a slot and is sized and shaped to accommodate a flow restrictor 250 to restrict flow passing through the distal passage 140 from the needle lumen. In an example, the flow restrictor 250 can embody a septum, a valve, or a hydrophobic filter or hydrophobic membrane. In use and upon accessing the patient's vasculature, blood pressure causes blood to flow proximally through the needle lumen. When the flow restrictor 250 is incorporated, proximal blood flow can be stopped or restricted at the flow restrictor, thereby eliminating or limiting blood leakage into the second section 232 and onto the working platform 136.

In an example, the receiving cavity 246 is sized and shaped to frictionally grip the flow restrictor 250 or hold the flow restrictor in an interference fit. The receiving cavity can be a slot and part of the receiving cavity can bisect the distal path 140 or the distal path can pass through or originates at the receiving cavity. In other examples, a retainer clip can be provided to snap over the opening of the receiving cavity 246 to secure the flow restrictor 250 therein. Preferably, the flow restrictor is sized and shaped to permit penetration by the guidewire and to permit movement of the guidewire therethrough during advancement of the guidewire.

With continued reference to FIG. 8B, the second section 232 is generally arc shape along a side view, in which the working surface 138 of the working platform 136 and the opposed bottom surface 254 have a matching arc-shape. The two surfaces 138, 254 are separated from one another by a thickness that defines a thickness of the needle hub. The thickness can be constant or can vary or non-constant along the length of the working platform. In other examples, the two surfaces 138, 254 can have different arcs. In still yet other examples, the bottom surface 254 can be flat or generally planar or can have a combination of planar and undulating surfaces.

The surface 138 of the working platform 136 is preferably arc-shape. The arc-shape surface helps to move the working surface 138 of the working platform 136 closer to the practitioner's finger as the practitioner uses his or her finger to slide the guidewire against the surface 138 from a proximal position to a distal position. However, the surface 138 is not limited to an arc shape. For example, the working surface 138 of the working platform 136 can be generally planar or can have different degrees of arc-shape. Thus, where the second section 232 can have two upper reference points 259, 261 defining a plane, the surface 138 of the working platform 232 can locate below the or subjacent the plane and can have an arc-shape.

In addition to the upper surface 138 and the bottom surface 254, the second section has two opposed sides 122. The sides can be smooth or flat or can have surface features 170, as previously discussed. The sides can alternatively have grooves to receive fingers of the holding hand.

The third section 234 of the needle hub 110 can optionally include a guide stub 150. The guide stub 150 can be unitarily formed, such as by single plastic injection molding, with the first and second sections 230, 232 or can be separately formed and secured to the first and second sections. For example, the guide stub 150 can be separately molded with a bore or passage and then glued, snap-fit, or welded to the proximal end surface 152 of the second section 232. If separately formed, the part of the proximal path 144 formed with the second 232 and the part of the proximal path formed with the guide stub 150 should align.

In an example, the guide stub 150 is generally cylindrical. In other examples, the guide stub can have multi-sides, such as having a polygonal shape. The guide stub extends the overall length of the proximal path 144 and can provide an anchor point or attachment point for a tubing, as previously discussed. However, the guide stub 150 may be omitted and the second section 232 can be provided with a sufficient path length and serves as an attachment point for the tubing. For example, from the end point 260 of the second section 232, the second section can be extended in the proximal direction so that the proximal end surface 152 is extended further proximally instead of coinciding with the end point 260. This allows the path 144 to be lengthened and the tubing can couple directly to the end opening thereof.

With reference now to FIG. 9, a cross-sectional side view of a needle hub 100 provided in accordance with further aspects of the invention is shown. The needle hub 100 is similar to the needle hub 100 of FIGS. 8A and 8B with a few exceptions. In the present example, the neck section 242 has been modified to have a generally cylindrical shape and the receiving cavity 246 has been modified to receive a relatively smaller flow restrictor 250. The flow restrictor 250 is located at the distal path opening 140a of the distal path 140 and gripped along at least two sides, preferably at least three sides, by the structure of the receiving cavity 246. Optionally, the flow restrictor 250 can be retained to the receiving cavity 246, such as with a clip, a detent engagement, by adhesive, or combinations thereof.

The second section 232 of the needle hub 110 comprises a working platform 136 having a surface 138 and a lower surface 254, which can both be arc-shaped like that of FIG. 8B. However, in the present embodiment, the working platform 136 resembles a bowl shape channel and the upper or working surface 138 of the working platform 136 is located at the bottom of the channel. The working platform 136 has two upwardly extending sides 264 (only one shown) that extend from the working surface 138. Along an end cross-section, the bowl shape channel resembles the letter “U”, but with relatively lower extending sidewalls 264 resembling a short U. Further, the channel arcs or curves as the surface 138 extends from the proximal end to the distal end. For example, the working surface 138 and the upper edge 268 of both sidewalls 264 have an arc-shape. The channel helps to locate the practitioner's finger between the two sidewalls 264 when using the catheter assembly to advance the guidewire.

FIG. 9 shows the guide stub 150 of the third section 234 as a separately formed component that is attached to the second section 232, such as by adhesive or welding. However, as previously discussed, the guide stub 150 can be singularly formed with the second section 232 or can be omitted altogether. If separately formed, then the first proximal path section 144(1) of the proximal path 144 aligns with the second proximal path section 144(2) of the proximal path 144 to define an overall proximal path 144. As shown, the upper contour of the guide stub 150 is generally flat or flush with the upper proximal point 260 of the second section 232. In other examples, the upper contour of the guide stub 150 can locate above or below with the upper proximal point of the second section.

The rear-most opening 144b of the proximal path 144 is preferably enlarged or is preferably larger than the diameter of the path itself. The rear-most opening 144b can be enlarged to facilitate insertion of the guidewire 148. The rear-most opening 144b can be enlarged to receiving a tubing 158, as shown with reference to FIG. 1. The tubing 158 can attach to the rear-most opening 144b via friction fit, interference fit, by welding, by adhesive, or combinations thereof.

The needle hub 100 of FIGS. 8A and 8B or of FIG. 9 may be used as described elsewhere herein, such as with reference to FIGS. 1, 4A, 5(A)-5(C), and 7(A)-7(D).

FIG. 10 shows a user 174 gripping the catheter assembly 100 with one hand in a supinated position as previously discussed. The catheter assembly 100 comprises a catheter unit 102 and a needle unit 104. The catheter unit 102 can embody any of the catheter units described elsewhere herein, such as the catheter unit of FIG. 4B. The needle unit 104 can embody any of the needle units described elsewhere herein, including the needle unit of FIGS. 1, 4A, 8A, 8B, and 9. The user 174 can hold the catheter assembly 100 with the holding hand 175, which can be a left hand or a right hand. The user 174 has punctured the patient 210 at a puncture site and the needle tip 114 and catheter tube opening 116 have entered the vein 212, which causes blood 272 to flow upward through the lumen of the needle 112, shown schematically for illustration purposes. In some examples, the needle unit 104 can have different ergonomic shapes than as shown to aid the user on where to grip the needle unit.

In an example, proximal blood flow through the needle is stopped or restricted by a flow restrictor 250. The flow restrictor 250 can be located in a receiving cavity formed with the needle hub 110, as discussed with reference to FIGS. 8A, 8B and 9. The flow restrictor 250, which can be a septum, a valve, or a hydrophobic filter, can restrict or limit blood flow from flowing onto and wetting the surface of the working platform 136. FIG. 10 resembles the position of FIG. 5(A) and FIG. 7(A) and may be used in combination with an ultrasound probe.

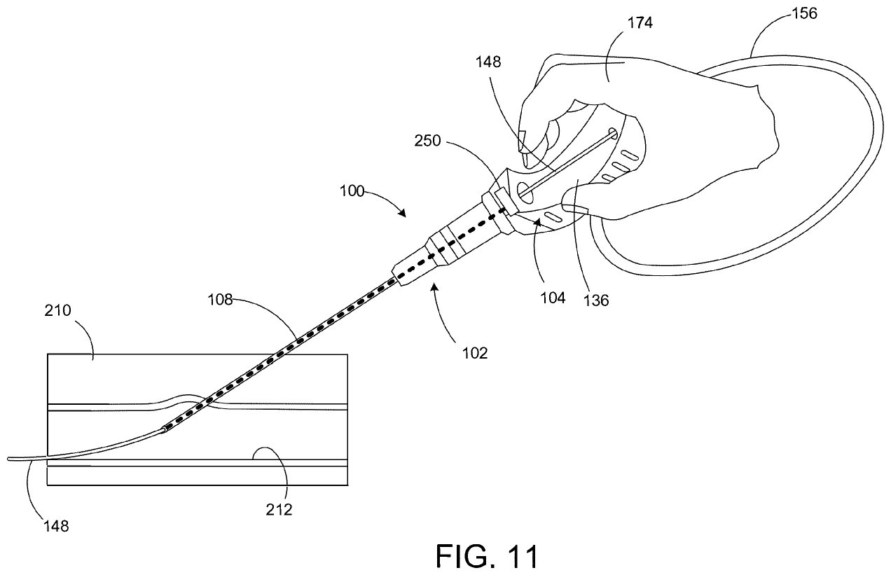

With reference now to FIG. 11, the catheter assembly 100 is shown with the guidewire 148 advanced distally out the needle tip 114 and into the vasculature of the patient 210. In the present embodiment, the guidewire 148 is advanced distally, which requires at least part of the guidewire to move through the flow restrictor 250 when advanced by the user. Because the flow restrictor 250 is incorporated with the integrated guidewire and needle hub, blood flowing through the needle lumen is restricted or blocked from spilling onto the working platform 136.

FIG. 11 resembles the position of FIGS. 5(B) and 7(B) and may be used in combination with an ultrasound probe.

Methods of making and of using the catheter assemblies and components thereof are within the scope of the present invention.

Although limited embodiments of the catheter assemblies and their components have been specifically described and illustrated herein, many modifications and variations will be apparent to those skilled in the art. Accordingly, it is to be understood that the catheter assemblies and their components constructed according to principles of the disclosed device, system, and method may be embodied other than as specifically described herein. The disclosure is also defined in the following claims.

1-20. (canceled)

21. A catheter assembly comprising:
a catheter unit comprising a catheter hub having a hub body with a proximal opening and having a catheter tube extending from a distal end of the catheter hub, the catheter tube having a catheter tube opening;
an integrated needle unit comprising a needle hub with a body and having a needle with a needle lumen extending out a distal end of the needle hub, wherein the needle hub comprises:
a distal path having a distal path opening and having the needle attached to an end of the distal path;
a proximal path having a proximal path opening and a rear-most opening;
a working surface on a working platform located between the distal path opening and the proximal path opening; and
wherein the working surface is exposed from a side of the body,
wherein a guidewire extends through both the distal path, the proximal path, and at least part of the needle lumen; and
wherein the assembly is configured such that, after advancement of the guidewire,
the catheter tube is configured to advance over the guidewire causing the catheter hub to separate from the needle hub such that the needle hub, the needle, and the guidewire are completely separated from the catheter hub, when the needle hub and the catheter hub are moved away from one another.

22. The catheter assembly of claim 21, wherein the proximal opening of the hub body of the catheter hub comprises a female Luer receiving a distal end of the needle hub.

23. The catheter assembly of claim 21, wherein a tubing having a first end is coupled directly to the needle hub.

24. The catheter assembly of claim 23, wherein the first end projects into the rear-most opening of the needle hub.

25. The catheter assembly of claim 23, wherein the guidewire is partly located inside the tubing.

26. The catheter assembly according to claim 23, wherein the tubing has a second end and wherein the second end is unsupported or wherein the second end is supported by a socket located on the needle hub.

27. The catheter assembly according to claim 21, wherein the guidewire has a proximal end and wherein the proximal end is sized and shaped to abut the rear-most opening of the needle hub to stop distal movement of the guidewire or wherein the guidewire has an enlarged section and wherein the enlarged section is sized and shaped to abut a crimp on the needle from inside the needle to stop distal movement of the guidewire.

28. The catheter assembly according to claim 21, wherein the needle hub has a first proximal path section and a guide stub extending from a proximal end surface of the needle hub and a second proximal path section aligned with the first proximal path section.

29. The catheter assembly according to claim 21, wherein the needle hub has a receiving cavity at the distal path.

30. The catheter assembly according to claim 21, further comprising a flow restrictor located in the receiving cavity, said flow restrictor comprising one of a septum, a valve, or a hydrophobic filter.

31. The catheter assembly according to claim 21, wherein the needle hub has a channel and wherein the working platform is part of the channel and the channel having sidewalls extending from the working surface.

32. The catheter assembly according to claim 21, further comprising a needle guard located in an interior of the catheter hub, said needle guard comprising two arms biased outwardly by the needle.

33. The catheter assembly of claim 32, further comprising a valve and a valve opener located in the interior of the catheter hub.

34. The catheter assembly according to claim 21, wherein a tubing is attached to the needle hub and wherein a guidewire is partly located inside the tubing and partly located inside the needle.

35. The catheter assembly of claim 34, wherein the guidewire passes through both the distal path and the proximal path.

36. A method of manufacturing a catheter assembly comprising:
forming a catheter unit comprising a catheter hub having a hub body with a proximal opening and having a catheter tube extending from a distal end of the catheter hub, the catheter tube having a catheter tube opening;
forming an integrated needle unit comprising a needle hub with a body and having a needle with a needle lumen extending out a distal end of the needle hub, wherein the needle hub comprises:
a distal path having a distal path opening and having the needle attached to an end of the distal path;
a proximal path having a proximal path opening and a rear-most opening;
a working surface on a working platform located between the distal path opening and the proximal path opening; and
wherein the working surface is exposed from a side of the body, and
passing a guidewire through proximal path, through the distal path, and at least partly into the needle lumen.

37. The method of claim 36, further comprising exposing the guidewire at the working surface between the proximal path and through the distal path for pushing by a user.

38. The method of claim 36, further comprising placing a flow restrictor into a receiving cavity formed at a distal end of the needle hub.

39. A method of using a catheter assembly comprising:
gripping the catheter assembly with one hand to grip the catheter assembly with a holding hand, the catheter assembly comprising:
a catheter unit comprising a catheter hub having a hub body with a proximal opening and having a catheter tube extending from a distal end of the catheter hub, the catheter tube having a catheter tube opening;
an integrated needle unit comprising a needle hub with a body and having a needle with a needle lumen extending out a distal end of the needle hub, wherein the needle hub comprises:
a distal path having a distal path opening and having the needle attached to an end of the distal path;
a proximal path having a proximal path opening and a rear-most opening;
a working surface on a working platform located between the distal path opening and the proximal path opening; and
wherein the working surface is exposed from a side of the body;
orienting a finger of the holding hand over the working platform.

40. The method of claim 39, wherein a guidewire passes between the distal path and the proximal path, and further comprising pushing down on the guidewire with the finger to press the guidewire between the finger and the working surface.
